SEQUENCE LISTING (14 sequences)

SEQ ID NO: 1 - DNA, 20 bp, synthetic construct
  source 1..20: mol_type = other DNA
ggctgccaagtgtcaaatcc

SEQ ID NO: 2 - DNA, 20 bp, synthetic construct
  source 1..20: mol_type = other DNA
agtgctcccccacttacttg

SEQ ID NO: 3 - DNA, 20 bp, synthetic construct
  source 1..20: mol_type = other DNA
cggagccctctttcacagtt

SEQ ID NO: 4 - DNA, 20 bp, synthetic construct
  source 1..20: mol_type = other DNA
ttcagcttaggagcgaaggc

SEQ ID NO: 5 - DNA, 20 bp, synthetic construct
  source 1..20: mol_type = other DNA
aggtgctgtagcgggatttc

SEQ ID NO: 6 - DNA, 20 bp, synthetic construct
  source 1..20: mol_type = other DNA
agaggcactgtatagggcag

SEQ ID NO: 7 - DNA, 20 bp, synthetic construct
  source 1..20: mol_type = other DNA
ctgctgacatgcttggaaaa

SEQ ID NO: 8 - DNA, 20 bp, synthetic construct
  source 1..20: mol_type = other DNA
attgggctaccccagcaatg

SEQ ID NO: 9 - DNA, 20 bp, synthetic construct
  source 1..20: mol_type = other DNA
agcgctcccccacttacttg

SEQ ID NO: 10 - DNA, 20 bp, synthetic construct
  source 1..20: mol_type = other DNA
gacagggacatcagtcgctt

SEQ ID NO: 11 - DNA, 20 bp, synthetic construct
  source 1..20: mol_type = other DNA
atgaatgaccactctagcca

SEQ ID NO: 12 - DNA, 20 bp, synthetic construct
  source 1..20: mol_type = other DNA
atagaaacagccaggaccgc

SEQ ID NO: 13 - DNA, 22 bp, synthetic construct
  source 1..22: mol_type = other DNA
gtggtctcctctgacttcaaca

SEQ ID NO: 14 - DNA, 22 bp, synthetic construct
  source 1..22: mol_type = other DNA
ctcttcctcttgtgctcttgct

(19) United States
(12) Patent Application Publication
(10) Pub. No.: US 2026/0355272 A1
(43) Pub. Date: Nov. 12, 2026

(54) COMPOSITION FOR PREVENTING, TREATING, OR AMELIORATING NEURODEGENERATIVE DISORDER, COMPRISING ALLIUM SENESCENS EXTRACT AS ACTIVE INGREDIENT

(71) Applicant: EVERBIO CO., LTD., Seoul (KR)

(72) Inventors: Boyong KIM, Gyeonggi-do (KR); Jae Hyeok JEONG, Gyeonggi-do (KR)

(21) Appl. No.: 19/125,597

(22) PCT Filed: Nov. 6, 2023
(86) PCT No.: PCT/KR2023/017686

§ 371 Date: Apr. 29, 2025

(30) Foreign Application Priority Data
Nov. 4, 2022 (KR) 10-2022-0146551
Mar. 21, 2023 (KR) 10-2023-0036968

(51) Int. Cl. A61K 36/8962; A61P 25/28

(52) U.S. Cl. CPC A61K 36/8962; A61P 25/28

(57) ABSTRACT

The present invention relates to a composition for preventing, treating, or ameliorating a neurodegenerative disorder, comprising an Allium senescens extract as an active ingredient. The composition of the present invention reduces the level of reactive oxygen species (ROS) in nerve cells, protects the mitochondrial activity of nerve cells, and inhibits mitochondrial apoptosis of nerve cells, and thus, the composition may be utilized to prevent, treat, or ameliorate a neurodegenerative disorder.

### TECHNICAL FIELD

This patent application claims the benefit of and priority to Korean Patent Application No. 10-2022-0146551, filed with the Korean Intellectual Property Office on Nov. 4, 2022, and Korean Patent Application No. 10-2023-0036968, filed with the Korean Intellectual Property Office on Mar. 21, 2023, the disclosures of which are incorporated herein by reference.

The present disclosure relates to a composition comprising Allium senescens extract as an active ingredient for the prevention, treatment, or alleviation of neurodegenerative diseases.

### BACKGROUND ART

Neurodegenerative disorders generally occur in elderly individuals aged 60 years or older. As modern society transitions into an aging society, the number of patients with neurodegenerative diseases is rapidly increasing, and interest in their prevention and treatment is growing day by day. Among neurodegenerative diseases, Parkinson's disease has exhibited the fastest-growing patient population since 1990, with the number of cases doubling to 6.2 million from 1990 to 2015. Parkinson's disease is a chronic progressive central nervous system disorder caused by dysfunction of dopamine-secreting neurons in the substantia nigra. Dopamine is an essential neurotransmitter in the brain and plays a crucial role in transmitting neuronal excitatory signals. Consequently, dopamine deficiency results in the loss of motor control.

Levodopa (L-Dopa) is the most commonly used drug for alleviating Parkinson's disease symptoms, as it is absorbed into the brain and converted into dopamine. However, in some patients, levodopa itself has been reported to cause severe side effects, such as levodopa-induced dyskinesia (LID). In addition to levodopa, various compounds, including dopamine agonists and monoamine oxidase B (MAOB) inhibitors, have been used to treat Parkinson's disease; however, these drugs are associated with side effects such as nausea, drowsiness, and vomiting.

To date, no Parkinson's disease treatment containing bioactive components extracted from natural substances has been commercially available. Accordingly, there is a growing unmet demand for pharmaceuticals and functional foods that exhibit excellent preventive, therapeutic, and ameliorative effects against Parkinson's disease while being free from side effects.

Throughout this specification, multiple academic papers and patent documents are cited and referenced. The disclosures of the cited papers and patent documents are incorporated herein by reference in their entirety to clarify the level of the technical field and further describe the present disclosure.

### DISCLOSURE OF INVENTION

### Technical Problem

Leading to the present disclosure, thorough and intensive research conducted by the present inventors with the aim of developing a composition derived from natural substances that exhibits low toxicity to the human body, minimizes side effects, and is capable of preventing, treating, or alleviating neurodegenerative diseases, resulted in the finding that an extract of Allium senescens reduces the level of reactive oxygen species (ROS) in neural cells, protects neural cells from mitochondrial dysfunction caused by ROS, and inhibits mitochondrial apoptosis in neural cells, thereby exhibiting neuroprotective effects.

Accordingly, an aspect of the present disclosure is to provide a composition including Allium senescens extract as an active ingredient for the prevention, treatment, or alleviation of neurodegenerative diseases.

Other features and advantages of the present disclosure will become more apparent from the following detailed description of the invention, the claims, and the drawings.

### Solution to Problem

Provided according to one aspect of the present disclosure is a pharmaceutical composition comprising Allium senescens extract as an active ingredient for the prevention or treatment of neurodegenerative diseases.

The inventors of the present disclosure have conducted extensive research to develop a composition derived from natural substances that exhibits low toxicity to the human body, minimizes side effects, and is capable of preventing, treating, or improving neurodegenerative diseases. As a result, the present inventors have identified that Allium senescens extract reduces the level of reactive oxygen species (ROS) in neural cells, protects neural cells from mitochondrial dysfunction caused by ROS, and inhibits mitochondrial apoptosis in neural cells, thereby exhibiting neuroprotective effects.

In this specification, Allium senescens refers to a perennial herbaceous plant of the Liliaceae family distributed in Northern Europe and Asia, particularly on Ulleungdo Island in Korea. Allium senescens is known to purify blood, regulate blood cholesterol levels, activate detoxification processes, and restore liver function. The young leaves of Allium senescens are edible, while its stems are used as diuretics or tonics. The Allium senescens used in the present disclosure may be raw Allium senescens, ground herbal material, dried herbal material, pulverized dried herbal material, or fermented Allium senescens, with dried Allium senescens being a more specific example, but with no limitations thereto. Furthermore, there are no restrictions on the procurement method of Allium senescens, and it may be cultivated or purchased commercially.

The Allium senescens extract used in the composition of the present disclosure may be an extract obtained from the aerial or root parts of Allium senescens, either in whole or in part. More specifically, the Allium senescens extract used in the composition of the present disclosure may be one or more extracts selected from the group consisting of leaves, flowers, stems, fruits, roots, and the whole herb of Allium senescens, with the extract from the leaves being a more specific example. However, the present disclosure is not limited thereto.

In an embodiment of the present disclosure, the Allium senescens extract used in the composition is an extract obtained from dried Allium senescens as described above. More specifically, the drying process may be carried out by spray drying, hot air drying, freeze drying, natural drying, or vacuum drying, but with no limitations thereto.

The Allium senescens extract used in the composition of the present disclosure may be obtained through direct extraction or commercial procurement. The extraction process may involve cutting or grinding Allium senescens to an appropriate size before extraction.

When the Allium senescens extract used in the composition of the present disclosure is obtained by treating Allium senescens with an extraction solvent, various extraction solvents such as polar and non-polar solvents may be used. Suitable polar solvents include (i) water; (ii) alcohols (preferably methanol, ethanol, propanol, butanol, n-propanol, isopropanol, n-butanol, 1-pentanol, 2-butoxyethanol, or ethylene glycol); (iii) acetic acid; (iv) dimethylformamide (DMFO); and (v) dimethyl sulfoxide (DMSO). Suitable non-polar solvents include acetone, acetonitrile, ethyl acetate, methyl acetate, fluoroalkanes, pentane, hexane, 2,2,4-trimethylpentane, decane, cyclohexane, cyclopentane, diisobutylene, 1-pentene, 1-chlorobutane, 1-chloropentane, o-xylene, diisopropyl ether, 2-chloropropane, toluene, 1-chloropropane, chlorobenzene, benzene, diethyl ether, diethyl sulfide, chloroform, dichloromethane, 1,2-dichloroethane, aniline, diethylamine, ether, carbon tetrachloride, and tetrahydrofuran (THF).

In an embodiment of the present disclosure, the Allium senescens extract is extracted using one extraction solvent selected from the group consisting of water, an organic solvent, and a mixed solvent thereof.

In an embodiment of the present disclosure, the organic solvent may be one or more selected from the group consisting of lower alcohols having 1 to 4 carbon atoms, petroleum ether, hexane, benzene, chloroform, methylene chloride, ether, ethyl acetate, and acetone, but with no limitations thereto.

The extraction temperature of the extract of the present disclosure is not particularly limited and may range, for example, from 0 °C to 140 °C. More specifically, the extraction temperature may be 0 °C to 140 °C, 0 °C to 120 °C, 0 °C to 100 °C, 0 °C to 85 °C, 0 °C to 75 °C, 0 °C to 70 °C, 20 °C to 140 °C, 20 °C to 120 °C, 20 °C to 100 °C, 20 °C to 85 °C, 20 °C to 75 °C, 20 °C to 70 °C, 40 °C to 140 °C, 40 °C to 120 °C, 40 °C to 100 °C, 40 °C to 85 °C, 40 °C to 75 °C, 40 °C to 70 °C, 55 °C to 140 °C, 55 °C to 120 °C, 55 °C to 100 °C, 55 °C to 85 °C, 55 °C to 75 °C, 55 °C to 70 °C, 65 °C to 140 °C, 65 °C to 120 °C, 65 °C to 100 °C, 65 °C to 85 °C, 65 °C to 75 °C, 65 °C to 70 °C, 70 °C to 140 °C, 70 °C to 120 °C, 70 °C to 100 °C, 70 °C to 85 °C, or 70 °C to 75 °C, but is not limited thereto.

The extraction time of the extract of the present disclosure is not particularly limited and may range, for example, from 10 minutes to 9 hours, 10 minutes to 6 hours, 10 minutes to 3 hours, 10 minutes to 120 minutes, 10 minutes to 100 minutes, 10 minutes to 90 minutes, 30 minutes to 9 hours, 30 minutes to 6 hours, 30 minutes to 3 hours, 30 minutes to 120 minutes, 30 minutes to 100 minutes, 30 minutes to 90 minutes, 45 minutes to 9 hours, 45 minutes to 6 hours, 45 minutes to 3 hours, 45 minutes to 120 minutes, 45 minutes to 100 minutes, 45 minutes to 90 minutes, 60 minutes to 9 hours, 60 minutes to 6 hours, 60 minutes to 3 hours, 60 minutes to 120 minutes, 60 minutes to 100 minutes, 60 minutes to 90 minutes, 75 minutes to 9 hours, 75 minutes to 6 hours, 75 minutes to 3 hours, 75 minutes to 120 minutes, 75 minutes to 100 minutes, or 75 minutes to 90 minutes. However, the present disclosure is not limited thereto.

The extract used in the present disclosure may be obtained through hot water extraction, cold immersion extraction, reflux cooling extraction, ultrasonic extraction, or other conventional extraction methods known in the art. The extraction process may be repeated from 1 to 10 times, more specifically, from 1 to 10 times, 1 to 7 times, 1 to 4 times, 1 to 3 times, 2 to 10 times, 2 to 7 times, 2 to 4 times, or 2 to 3 times.

In the present disclosure, the Allium senescens extract may be used in the form of a crude extract obtained by solvent extraction, and it may also be used after being highly purified.

As used herein, the term “extract” generally refers to a crude extract, as commonly understood in the art. However, in a broader sense, it also includes fractions obtained by further fractionation of the extract. That is, the Allium senescens extract includes not only the extract obtained using the extraction solvents described above but also extracts subjected to additional purification processes. For example, fractions obtained through various purification processes additionally conducted, such as fractions obtained by passing through an ultrafiltration membrane with a specific molecular weight cut-off value, fractions obtained through various chromatographic separation techniques (such as size-exclusion, ion-exchange, hydrophobic interaction, or affinity chromatography), etc. fall within the scope of the Allium senescens extract of the present disclosure.

The Allium senescens extract used in the present disclosure may be processed into powder form through additional processes such as vacuum distillation, freeze drying, or spray drying.

As used in this specification, the term “neurodegenerative disease” refers to abnormalities in motor control, cognitive function, perception, sensory function, and autonomic nervous system function caused by a decline in neural function or neuronal cell death. The term “degenerative brain disease” is used synonymously.

Neurodegenerative diseases may be classified based on clinical characteristics.

Diseases characterized primarily by progressive cognitive impairment include Alzheimer's disease, dementia (such as frontotemporal degeneration and Lewy body dementia), and corticobasal degeneration. Diseases characterized primarily by progressive motor ataxia include Parkinson's disease, multiple system atrophy, Huntington's disease, and progressive supranuclear palsy. Neurodegenerative diseases characterized by muscle weakness and muscle atrophy include amyotrophic lateral sclerosis (ALS) and spinal muscular atrophy (SMA).

In an embodiment of the present disclosure, the neurodegenerative diseases described above may be selected from the group consisting of Parkinson's disease, Alzheimer's disease, Huntington's disease, mild cognitive impairment, cerebral amyloid angiopathy, Down syndrome, amyloid-related stroke, systemic amyloidosis, senile dementia, amyotrophic lateral sclerosis (ALS), spinocerebellar atrophy, Tourette's syndrome, Friedrich's ataxia, Lewy body dementia, progressive supranuclear palsy, and frontotemporal dementia, but with no limitations thereto.

As used herein, the term “neuronal cell death” refers to damage or death of neuronal cells or neural tissues caused by various factors, including metabolic, toxic, neurotoxic, and chemical causes.

The neurodegenerative diseases that are the target of prevention, treatment, or alleviation by the composition of the present disclosure are accompanied by neuronal cell death. This neuronal cell death includes necrosis and apoptosis of neuronal cells.

As demonstrated in the following examples, the composition of the present disclosure reduces reactive oxygen species (ROS) levels in neuronal cells, protects mitochondrial activity in neuronal cells, and inhibits mitochondrial apoptosis in neuronal cells, thereby exhibiting neuroprotective effects.

As used herein, the term “prevention” refers to any action that inhibits or delays the onset of neurodegenerative diseases by administering a composition comprising Allium senescens extract as an active ingredient.

As used herein, the term “treatment” refers to any action that improves or beneficially alters the symptoms of neurodegenerative diseases by administering a composition comprising Allium senescens extract as an active ingredient.

The pharmaceutical composition of the present disclosure includes a pharmaceutically effective amount of the complex extract. The term “pharmaceutically effective amount” refers to an amount sufficient to achieve the efficacy or activity of the complex extract described above and is not particularly limited as long as it achieves the objectives of the present disclosure.

The pharmaceutical composition of the present disclosure includes a pharmaceutically acceptable carrier. The pharmaceutically acceptable carrier included in the pharmaceutical composition of the present disclosure is conventionally used in formulations and may include, but is not limited to, lactose, dextrose, sucrose, sorbitol, mannitol, starch, acacia gum, calcium phosphate, alginate, gelatin, calcium silicate, microcrystalline cellulose, polyvinylpyrrolidone, water, syrup, and mineral oil. In addition to these components, the pharmaceutical composition of the present disclosure may further include lubricants, humectants, sweeteners, flavoring agents, emulsifiers, suspending agents, and preservatives. Suitable pharmaceutically acceptable carriers and formulations are described in detail in Remington's Pharmaceutical Sciences (19th ed., 1995).

The pharmaceutical composition of the present disclosure may be administered orally or parenterally. In the case of parenteral administration, it may be administered via intravenous injection, subcutaneous injection, or intramuscular injection.

The appropriate dosage of the pharmaceutical composition of the present disclosure may vary depending on factors such as the formulation method, mode of administration, age, weight, gender, pathological condition, food intake, administration time, route of administration, excretion rate, and patient responsiveness. The general dosage of the pharmaceutical composition of the present disclosure ranges from 0.001 to 1000 mg/kg for adults. The human dosage may also be estimated based on animal experiments.

The pharmaceutical composition of the present disclosure may be prepared as a unit dosage form or incorporated into a multi-dose container by formulating it using a pharmaceutically acceptable carrier and/or excipient according to methods readily known to those skilled in the art. The formulation may be in the form of a solution, suspension, syrup, or emulsion in an oil or aqueous medium, or in the form of an extract, powder, granule, tablet, or capsule. Additionally, it may further include dispersing agents or stabilizers.

As used in this specification, “6-hydroxydopamine” (6-OHDA) is a type of neurotoxin known to induce Parkinson's disease. When neuronal cells are exposed to 6-OHDA, oxidative stress leads to pathological characteristics such as neurodegeneration, inflammation, and apoptosis. 6-OHDA enters dopaminergic and noradrenergic neurons, inhibiting neurotransmitter reuptake. As neurons are destroyed, reactive oxygen species (ROS), such as superoxide, accumulate in neurons.

In an embodiment of the present disclosure, the composition reduces reactive oxygen species (ROS) levels in neuronal cells. 6-OHDA triggers ROS production in neuronal cells, and as demonstrated in the following examples, treatment with Allium senescens extract significantly reduced ROS levels in neuronal cells.

In an embodiment of the present disclosure, the composition protects mitochondrial activity in neuronal cells. As demonstrated in the following examples, while 6-OHDA treatment inhibits mitochondrial activity, treatment with Allium senescens extract preserved mitochondrial activity in neuronal cells.

In an embodiment of the present disclosure, the composition inhibits mitochondrial apoptosis in neuronal cells. As demonstrated in the following examples, treatment with Allium senescens extract significantly reduced 6-OHDA-induced mitochondrial apoptosis.

In an embodiment of the present disclosure, the composition inhibits the expression of monoamine oxidase B (MAOB) and caspase-3 (Cas3) in neuronal cells. As demonstrated in the following examples, treatment with Allium senescens extract significantly reduced 6-OHDA-induced expression of MAOB and Cas3. [0042]6-OHDA stimulates ROS production in neuronal cells and promotes the expression of monoamine oxidase A (MAOA) and monoamine oxidase B (MAOB). MAOB generates ROS, and its expression has been reported to increase with aging. When MAOB is overexpressed in the brain, it degrades amyloid precursor protein (APP), leading to the accumulation of amyloid-beta (Aβ) peptides through the γ-secretase mechanism. This accumulation is a known cause of amyloid plaques, which are observed in Parkinson's disease and Alzheimer's disease. Therefore, the composition of the present disclosure effectively prevents, treats, or ameliorates Parkinson's disease and Alzheimer's disease by inhibiting MAOB expression.

Caspase-3 (Cas3) plays a key role in apoptosis by interacting with caspase-8 (Cas8) and caspase-9 (Cas9) to induce apoptosis. Additionally, Cas3 promotes the degradation of amyloid-beta precursor 4A, generating amyloid-beta (Aβ) peptides, which contribute to the formation of amyloid plaques observed in Parkinson's disease and Alzheimer's disease. Therefore, the composition of the present disclosure effectively prevents, treats, or ameliorates Parkinson's disease and Alzheimer's disease by inhibiting Cas3 expression.

In an embodiment of the present disclosure, the composition inhibits the expression of apoptosis markers. More specifically, the apoptosis markers may include, but are not limited to, monoamine oxidase B (MAOB), caspase-3 (Cas3), Bax, cytochrome C (CytC), or a combination thereof. Under cellular stress conditions such as DNA damage, hypoxia, UV exposure, or exposure to chemical substances such as ROS, mitochondrial activity in neuronal cells triggers the assembly of Bax protein, leading to the release of cytochrome C (CytC). The released CytC, along with Cas3-activated Cas9, promotes neuronal apoptosis.

In an embodiment of the present disclosure, the composition promotes the expression of anti-apoptotic markers. More specifically, the anti-apoptotic markers may include AKT (protein kinase B), P50 (nuclear factor kappa-light-chain-enhancer of activated B cells-P50, NFκB-P50), P52, BCL-2 (BCL2 apoptosis regulator), or a combination thereof, but are not limited thereto.

As demonstrated in the following examples, the composition of the present disclosure inhibited the expression of apoptosis markers Cas3, Bax, and CytC, while promoting the expression of anti-apoptotic markers AKT, P50, P52, and BCL-2.

Provided according to another aspect of the present disclosure is a food composition including Allium senescens extract as an active ingredient for the prevention or alleviation of neurodegenerative diseases.

As used in this specification, the term “alleviation” refers to any action that reduces the severity of neurodegenerative diseases through the administration of a composition comprising Allium senescens extract as an active ingredient.

When the composition of the present disclosure is formulated as a food composition, it may include ingredients that are conventionally added during food production, such as proteins, carbohydrates, fats, nutrients, seasonings, and flavoring agents. Examples of carbohydrates include monosaccharides such as glucose and fructose; disaccharides such as maltose, sucrose, and oligosaccharides; and polysaccharides such as dextrin and cyclodextrin, as well as sugar alcohols such as xylitol, sorbitol, and erythritol. Both natural and synthetic flavoring agents may be used as flavoring agents. For example, when the food composition of the present disclosure is formulated as a drink, additional ingredients such as citric acid, high-fructose corn syrup, sugar, glucose, acetic acid, malic acid, and fruit juice may be included along with the active ingredient of the present disclosure.

In the case of the food composition of the present disclosure, the complex herbal extract included as an active ingredient in the pharmaceutical composition, as described in the above aspect of the present disclosure, may be used as is. Therefore, to avoid excessive complexity in this specification, redundant descriptions are omitted.

Provided according to another aspect of the present disclosure is a method for preventing or treating neurodegenerative diseases, the method including a step of administering to a subject a pharmaceutical composition including the aforementioned Allium senescens extract as an active ingredient.

As used herein, the term “administer” or “administration” refers to the direct introduction of a therapeutically or prophylactically effective amount of the composition of the present disclosure to a subject (individual) experiencing or at risk of experiencing the target disease, thereby ensuring that the same amount is formed within the subject's body.

The term “therapeutically effective amount” refers to an amount sufficient to provide a therapeutic or prophylactic effect to the subject receiving the composition, and it includes a “prophylactically effective amount”. Furthermore, as used herein, the term “subject” (individual) refers to mammals, including humans, mice, rats, guinea pigs, dogs, cats, horses, cattle, pigs, monkeys, chimpanzees, baboons, and rhesus macaques. More specifically, the subject of the present disclosure is a human.

Since the method for preventing, alleviating, or treating neurodegenerative diseases in the present disclosure includes the step of administering either the pharmaceutical composition or food composition of the present disclosure, redundant descriptions are omitted to avoid excessive repetition in this specification.

### Advantageous Effects of Invention

The features and advantages of the present disclosure can be summarized as follows:
(a) The present disclosure provides a composition for the prevention, treatment, or alleviation of neurodegenerative diseases.
(b) The composition for the prevention, treatment, or alleviation of neurodegenerative diseases in the present disclosure reduces the level of reactive oxygen species (ROS) in neuronal cells, protects mitochondrial activity in neuronal cells, and inhibits mitochondrial apoptosis, thereby making it useful for the prevention, treatment, or alleviation of neurodegenerative diseases.

### BRIEF DESCRIPTION OF DRAWINGS

FIG. 1a shows the viability of neuroblastoma cells according to the concentration of 6-OHDA treatment. (P<0.05)

FIG. 1b shows the viability of neuroblastoma cells according to the concentration of Allium senescens extract treatment. (P<0.05)

FIG. 2a shows the apoptosis of neuroblastoma cells according to the presence or absence of Allium senescens extract treatment.

FIG. 2b shows the relative proliferation of surviving neuroblastoma cells according to the presence or absence of Allium senescens extract treatment (Con represents the control group, **P<0.01, ***P<0.001).

FIG. 3a shows the results of mitochondrial membrane potential measurements using a fluorescence microscope, where the green-stained regions indicate apoptotic cells. (The white bar represents 20 μm.)

FIG. 3b shows the results of mitochondrial membrane potential measurements using a fluorescence microscope, where the red-stained regions indicate surviving cells. (The white bar represents 20 μm.)

FIG. 3c shows the number of inactivated cells counted by flow cytometry.

FIG. 3d presents a histogram showing the number of activated cells. (Con represents the control group, **P<0.01)

FIG. 4a shows the amount of ROS generated under different 6-OHDA and Allium senescens extract treatment conditions. (Con represents the control group, ns indicates no significant difference, *P<0.05, **P<0.01, ***P<0.001)

FIG. 4b shows the relative ROS generation under different 6-OHDA and Allium senescens extract treatment conditions. (*P<0.05, **P<0.01, ***P<0.001)

FIG. 5a shows the evaluation of monoamine oxidase B (MAOB) expression under different 6-OHDA and Allium senescens extract treatment conditions using flow cytometry. (*P<0.05, **P<0.01, ***P<0.001)

FIG. 5b shows the evaluation of caspase-3 (Cas3) expression under different 6-OHDA and Allium senescens extract treatment conditions using flow cytometry. (*P<0.05, **P<0.01, ***P<0.001)

FIG. 6 shows the mRNA levels of apoptosis markers in neuroblastoma cells under different 6-OHDA and Allium senescens extract treatment conditions. (Con represents the control group, ns indicates no significant difference, *P<0.05, **P<0.01, ***P<0.001)

FIG. 7a and FIG. 7b show the TUNEL assay results for the central nervous system (CNS) and ventral nervous system (VNS) of brine shrimp under different 6-OHDA and Allium senescens extract treatment conditions. (Con represents the control group, ns indicates no significant difference, *P<0.05, **P<0.01)

FIG. 7c shows the whole-mount immunohistochemistry results for the central and ventral nervous system cells of brine shrimp under different 6-OHDA and Allium senescens extract treatment conditions. (Con represents the control group, ns indicates no significant difference, *P<0.05, **P<0.01)

All experiments were conducted in triplicate (n=3). Data analysis was performed using SPSS software v26 (IBM, USA) and Prism 7 (GraphPad, USA). Statistical analysis was conducted using one-way analysis of variance (ANOVA), post hoc tests (Scheffe's method), and independent t-tests.

### BEST MODE FOR CARRYING OUT THE INVENTION

A better understanding of the present disclosure may be obtained through the following Examples, which are set forth to illustrate, but are not to be construed to limit, the present disclosure. It will be apparent to those skilled in the art that the scope of the present disclosure is not limited by these examples.

### EXAMPLES

(Throughout this specification, unless otherwise stated, the “%” used to indicate the concentration of a specific substance refers to (weight/weight) % for solid/solid, (weight/volume) % for solid/liquid, and (volume/volume) % for liquid/liquid.)

### Preparation Example 1: Preparation of Allium Senescens Extract

Allium senescens was purchased from a cultivated source on Ulleungdo Island and used in the experiment. The leaves of Allium senescens were dried, ground to 35 mesh, hydrated for 90 minutes, and then subjected to hot water extraction at 70 °C twice. The extracted solution was then filtered and concentrated using an R-3000 concentrator (BuCHI Labortechnik AG, Germany). The concentrated extract was freeze-dried at 60 °C using an FD8505 freeze dryer (Ilshin Lab Co., Korea).

### Example 1: Assay for Cytotoxicity of Allium senescens Extract

### Example 1-1: Culturing SH—SY5Y Cells

Human neuroblastoma SH—SY5Y cells were purchased from the Korean Cell Line Bank. The SH—SY5Y cells were cultured in Dulbecco's Modified Eagle's Medium (DMEM) containing 10% heat-inactivated fetal bovine serum (FBS), 100 μg/mL penicillin, and 100 μg/mL streptomycin at 37 °C under 5% CO₂ conditions.

### Example 1-2: Assay for Cytotoxicity of Allium senescens Extract and 6-OHDA

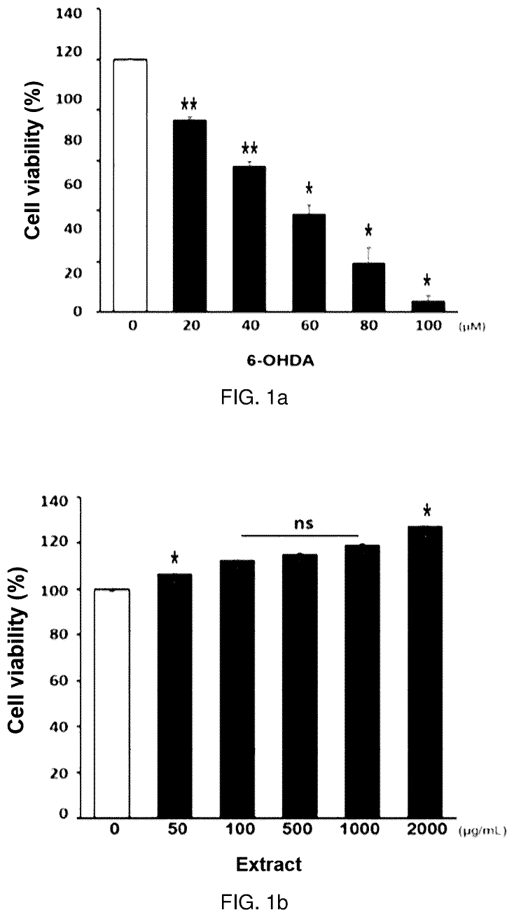

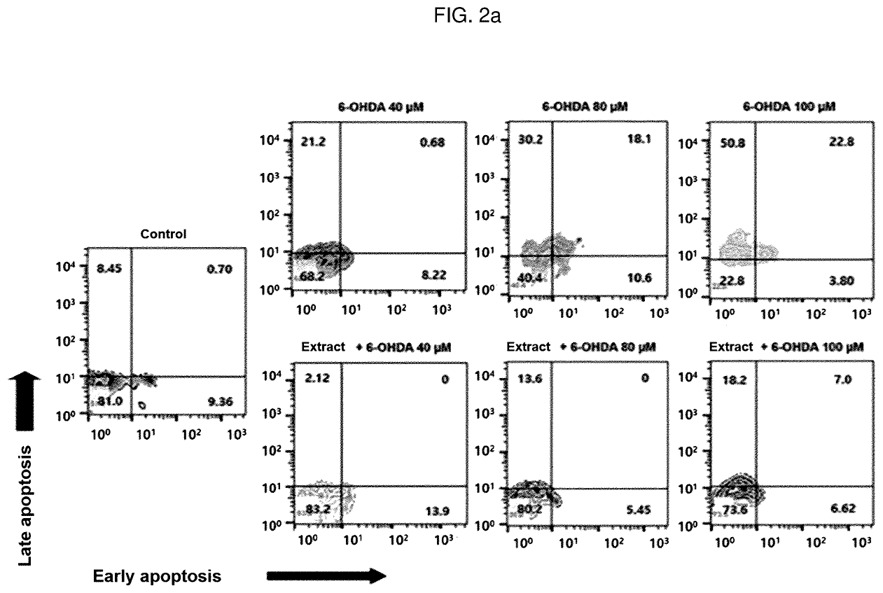

The SH—SY5Y cells cultured in Example 1-1 were exposed to 6-hydroxydopamine (6-OHDA) (Sigma-Aldrich, 162957) at concentrations of 0 μM, 20 μM, 40 μM, 60 μM, 80 μM, and 100 μM, and also to the Allium senescens extract prepared in Preparation Example 1 at concentrations of 50 μg/mL, 100 μg/mL, 500 μg/mL, 1000 μg/mL, and 2000 μg/mL for 12 hours. The results of the cell viability evaluation are shown in FIGS. 1a and 1b.

As shown in FIG. 1a, treatment with 6-OHDA led to a decrease in cell viability, with a sharp decline observed at concentrations exceeding 60 μM. In contrast, as shown in FIG. 1b, Allium senescens extract did not exhibit cytotoxicity in SH—SY5Y cells, confirming that Allium senescens extract possesses safety for use as a pharmaceutical or food composition for the prevention, treatment, or alleviation of neurodegenerative diseases.

Furthermore, based on the above results and the experimental data from Example 6, which demonstrated that AKT (Protein kinase B) expression remained constant at concentrations of 500 μg/mL or higher, 500 μg/mL of Allium senescens extract was selected for subsequent experiments.

In the following experiments, when Allium senescens extract was co-treated with 6-OHDA, the SH—SY5Y cells were first treated with 500 μg/mL of Allium senescens extract, followed by exposure to 6-OHDA at various concentrations for 1 hour.

### Example 2: Assay for Inhibitory Effect of Allium senescens Extract on Oxidative Apoptosis

To verify the oxidative apoptosis inhibitory effect of Allium senescens extract, the inventors conducted the following experiment.

The neuroblastoma cells cultured using the method described in Example 1-1 were divided into the following groups: i) Control group (Con): No treatment, ii) 6OHDA(40 μM) group: Treated with 40 μM 6-OHDA, iii) 6OHDA(40 μM)+EX group: Treated with 40 μM 6-OHDA and 500 μg/mL Allium senescens extract, iv) 6OHDA(80 μM) group: Treated with 80 μM 6-OHDA, v) 6OHDA(80 μM)+EX group: Treated with 80 μM 6-OHDA and 500 μg/mL Allium senescens extract, vi) 6OHDA(100 μM) group: Treated with 100 μM 6-OHDA, vii) 6OHDA(100 μM)+EX group: Treated with 100 μM 6-OHDA and 500 μg/mL Allium senescens extract. The degree of cell apoptosis was assessed using an Annexin-V-PI staining kit (Invitrogen, USA). In groups treated with both Allium senescens extract and 6-OHDA, the cells were pretreated with the extract for 1 hour, followed by 6-OHDA exposure for 1 hour.

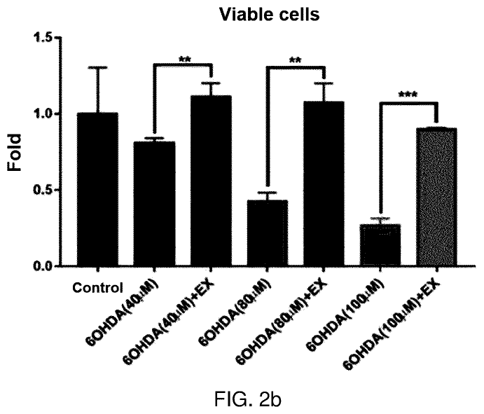

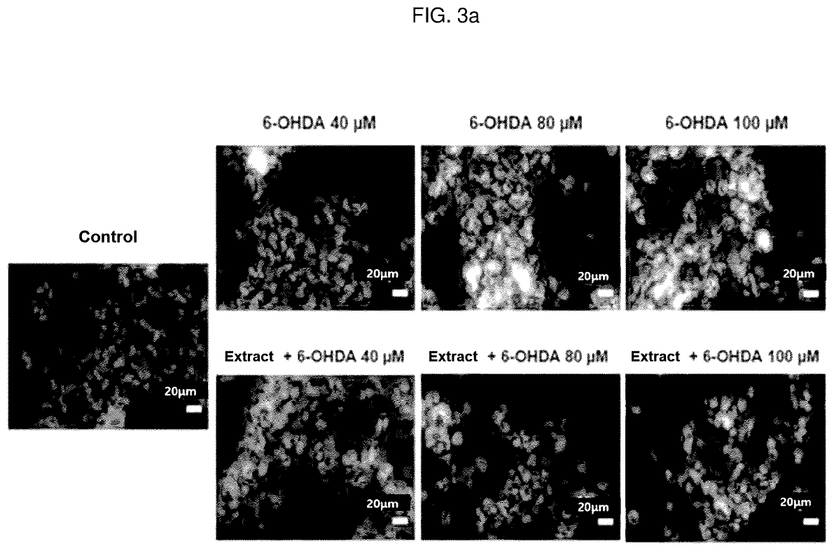

Annexin-V binds to phosphatidylserine (PS), which is externalized on the membrane of early apoptotic cells, allowing for early apoptosis detection. Propidium iodide (PI), which binds to the nucleus, stains cells in late apoptosis or necrosis by penetrating damaged cell membranes through holes formed therein. Apoptotic cells exhibit green fluorescence due to Annexin-V-FITC (Fluorescein isothiocyanate). Dead cells exhibit both red and green fluorescence due to PI uptake. Viable cells show minimal fluorescence. Cell viability was measured using a BD FACScalibur flow cytometer and FlowJo 10.6.1 (BD Bioscience), with the results shown in FIGS. 2a and 2b.

As shown in FIG. 2a, treatment with 6-OHDA alone significantly induced apoptosis in neuroblastoma cells, whereas co-treatment with Allium senescens extract reduced apoptosis (FIG. 2a). Furthermore, as described in Example 1-2, treatment with 6-OHDA alone at concentrations exceeding 60 μM caused a sharp decrease in cell viability. However, when co-treated with Allium senescens extract, cell viability remained high even when 6-OHDA concentrations exceeded 80 μM (FIG. 2b). Notably, when treated with 100 μM 6-OHDA alone (6OHDA(100 μM)), the relative number of surviving cells was 0.26-fold. However, when treated with 100 μM 6-OHDA and 500 μg/mL Allium senescens extract (6OHDA(100 μM)+EX), the relative survival was 0.91-fold, indicating that Allium senescens extract significantly increased cell survival.

These results confirm that Allium senescens extract effectively inhibits oxidative apoptosis induced by 6-OHDA in neuroblastoma cells.

### Example 3: Assay for Mitochondrial Activity Protection and Inhibition by Allium senescens Extract Against Mitochondrial Apoptosis

The present inventors conducted an experiment to evaluate the effect of Allium senescens extract on mitochondrial activity protection and mitochondrial apoptosis inhibition by measuring the mitochondrial membrane potential (MMP) under the following conditions.

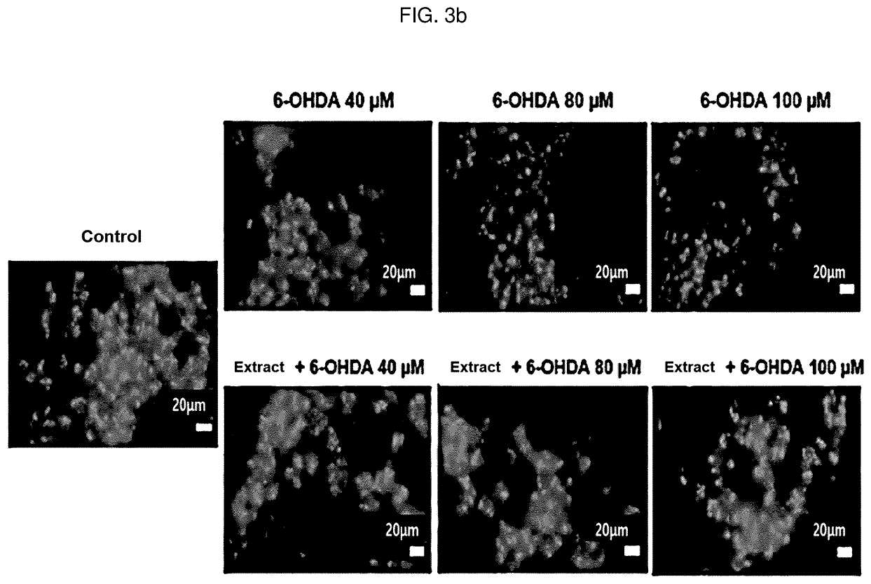

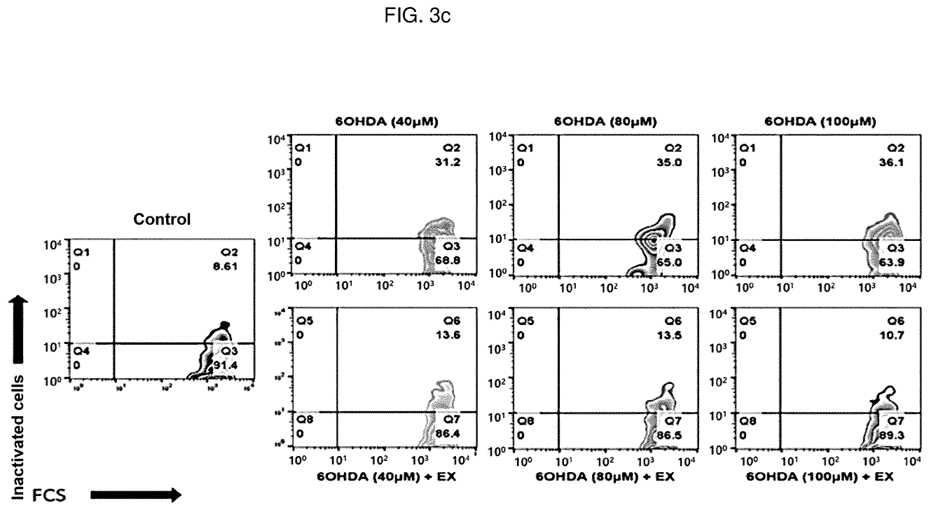

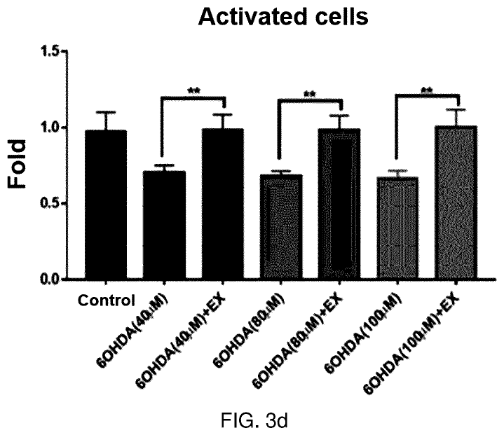

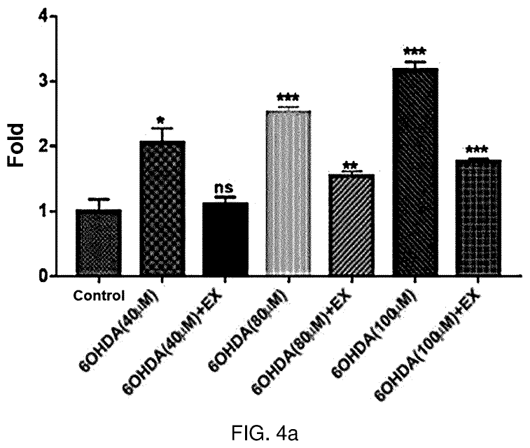

Neuroblastoma cells cultured according to Example 1-1 were divided into the following groups: i) Control group (Con): No treatment, ii) 6OHDA(40 μM) group: Treated with 40 μM 6-OHDA, iii) 6OHDA(40 μM)+EX group: Treated with 40 μM 6-OHDA and 500 μg/mL Allium senescens extract, iv) 6OHDA(80 μM) group: Treated with 80 μM 6-OHDA, v) 6OHDA(80 μM)+EX group: Treated with 80 μM 6-OHDA and 500 μg/mL Allium senescens extract, vi) 6OHDA(100 μM) group: Treated with 100 μM 6-OHDA, vii) 6OHDA(100 μM)+EX group: Treated with 100 μM 6-OHDA and 500 μg/mL Allium senescens extract. The cells were exposed to each treatment for three days, stained using a JC-1 Mitochondrial Membrane Potential Assay Kit (Invitrogen, USA), and mitochondrial activity was measured using a BD FACScalibur flow cytometer and FlowJo 10.6.1 (BD Bioscience). The results are shown in FIGS. 3a-3d.

The results demonstrated that mitochondrial activity was reduced by approximately 30% following 6-OHDA treatment (FIGS. 3a, 3c, and 3d). However, when 6-OHDA and Allium senescens extract were co-treated, mitochondrial activity was preserved (FIGS. 3b, 3c, and 3d).

These findings confirm that Allium senescens extract effectively inhibits mitochondrial apoptosis induced by 6-OHDA in neuroblastoma cells.

### Example 4: Assay for Reactive Oxygen Species (ROS) Reduction by Allium senescens Extract

The inventors conducted an experiment to evaluate the effect of Allium senescens extract on reactive oxygen species (ROS) levels in neuroblastoma cells. ROS are known to be toxic to proteins, lipids, carbohydrates, and DNA. Furthermore, accumulation of ROS in the central nervous system accelerates neurodegeneration and aging, contributing to diseases such as Alzheimer's and Parkinson's disease.

Neuroblastoma cells cultured using the method in Example 1-1 were divided into the following groups: i) Control group (Con): No treatment, ii) 6OHDA(40 μM) group: Treated with 40 μM 6-OHDA, iii) 6OHDA(40 μM)+EX group: Treated with 40 μM 6-OHDA and 500 μg/mL Allium senescens extract, iv) 6OHDA(80 μM) group: Treated with 80 μM 6-OHDA, v) 6OHDA(80 μM)+EX group: Treated with 80 μM 6-OHDA and 500 μg/mL Allium senescens extract, vi) 6OHDA(100 μM) group: Treated with 100 μM 6-OHDA, vii) 6OHDA(100 μM)+EX group: Treated with 100 μM 6-OHDA and 500 μg/mL Allium senescens extract. When applying both Allium senescens extract and 6-OHDA thereto, the group was treated with Allium senescens extract for 1 hour before exposure to 6-OHDA for a duration of 1 hour.

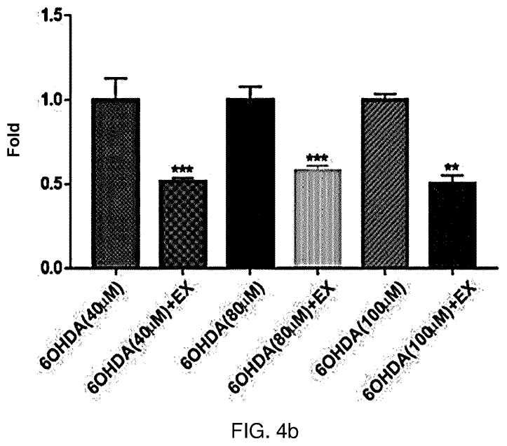

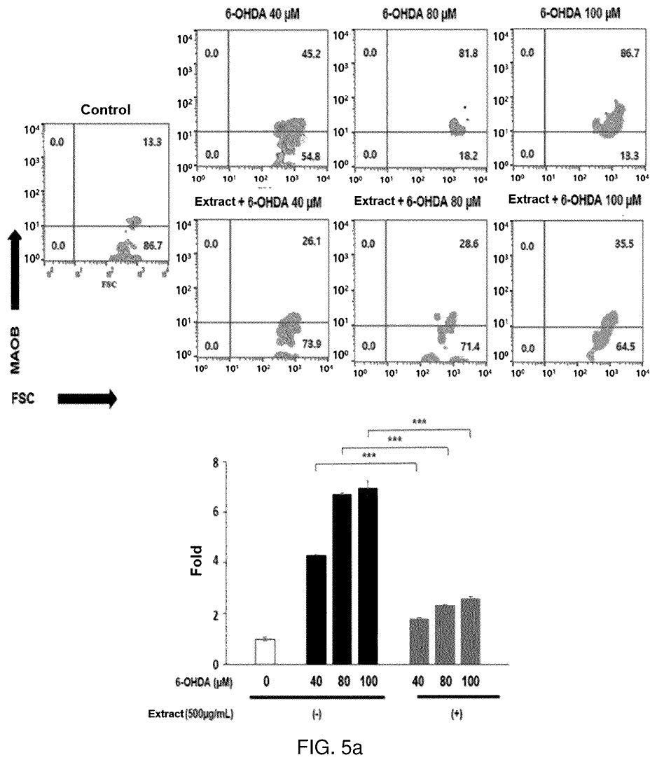

The cells were stained using a DCFDA-Cellular ROS Assay Kit (Abcam, UK) for 30 minutes, and ROS levels were measured using a BD FACScalibur flow cytometer and FlowJo 10.6.1 (BD Bioscience). The results are shown in FIGS. 4a and 4b.

As a result, 6-OHDA treatment increased ROS production (FIG. 4a). However, when 6-OHDA and Allium senescens extract were co-treated, ROS production was significantly reduced (FIG. 4b).

These findings confirm that Allium senescens extract reduces ROS levels in neuroblastoma cells, thereby: protecting mitochondrial activity in neuroblastoma cells; inhibiting mitochondrial apoptosis in neuroblastoma cells, and potentially preventing neurodegenerative diseases.

### Example 5: Assay for Mitochondrial Apoptosis-Related Marker Expression by Allium senescens Extract

The inventors conducted an experiment to evaluate the effect of Allium senescens extract on the expression of mitochondrial apoptosis-related markers in neuronal cells, specifically monoamine oxidase B (MAOB) and caspase-3 (Cas3) in neuroblastoma cells.

Neuroblastoma cells cultured using the method in Example 1-1 were divided into the following groups: i) Control group (Con): No treatment, ii) 6OHDA(40 μM) group: Treated with 40 μM 6-OHDA, iii) 6OHDA(40 μM)+EX group: Treated with 40 μM 6-OHDA and 500 μg/mL Allium senescens extract, iv) 6OHDA(80 μM) group: Treated with 80 μM 6-OHDA, v) 6OHDA(80 μM)+EX group: Treated with 80 μM 6-OHDA and 500 μg/mL Allium senescens extract, vi) 6OHDA(100 μM) group: Treated with 100 μM 6-OHDA, vii) 6OHDA(100 μM)+EX group: Treated with 100 μM 6-OHDA and 500 μg/mL Allium senescens extract. When applying both Allium senescens extract and 6-OHDA thereto, the group was treated with Allium senescens extract for 1 hour before exposure to 6-OHDA for 1 hour.

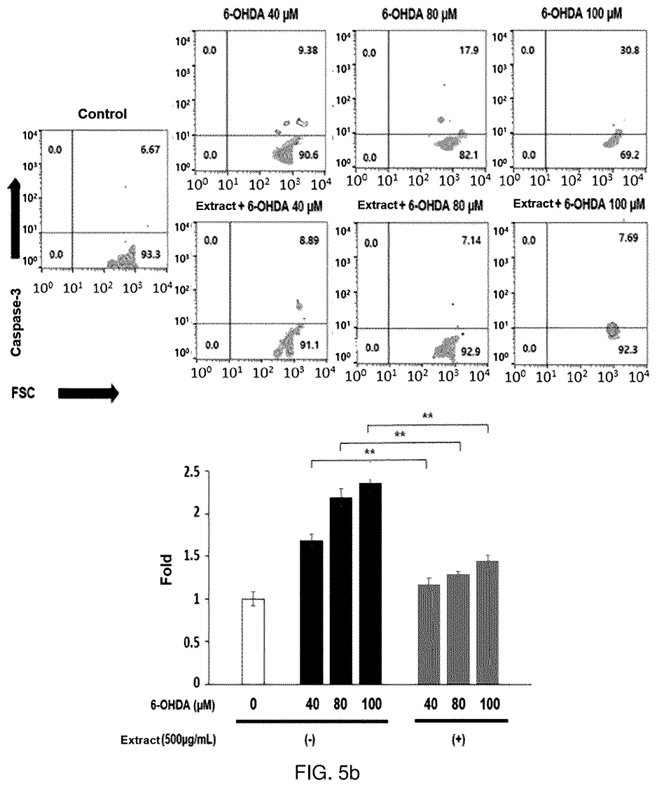

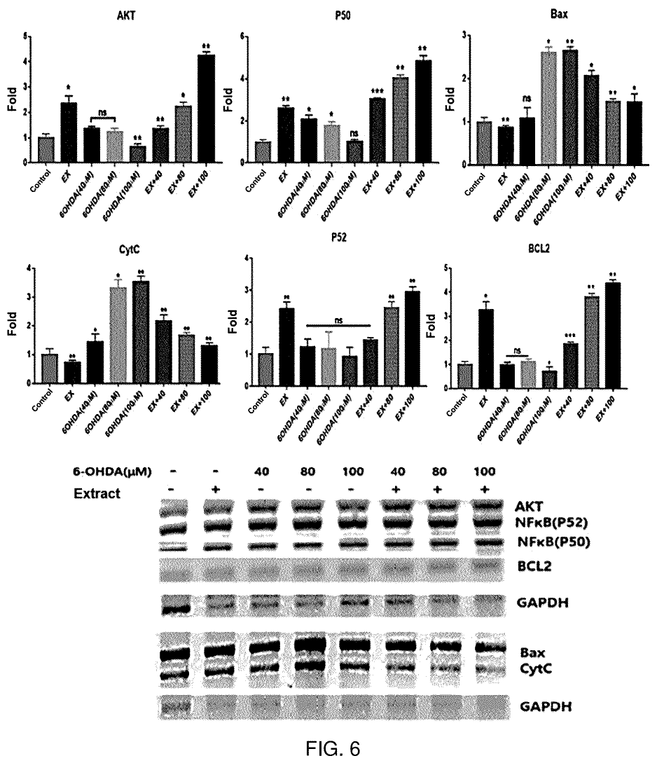

All cell samples were fixed in 2% paraformaldehyde for 4 hours, treated with 0.02% Tween 20 for 5 minutes, and blocked using Fc-blocking solution (BD Bioscience). The samples were incubated for two days with: FITC-anti-MAOB (Biocompare, USA) and Alexa Fluor 680-anti-Cas3 (Santa Cruz Biotechnology, USA). The samples were washed with phosphate-buffered saline (PBS) and analyzed using a BD FACScalibur flow cytometer and FlowJo 10.7.0 (BD Bioscience). The results are shown in FIGS. 5a and 5b.

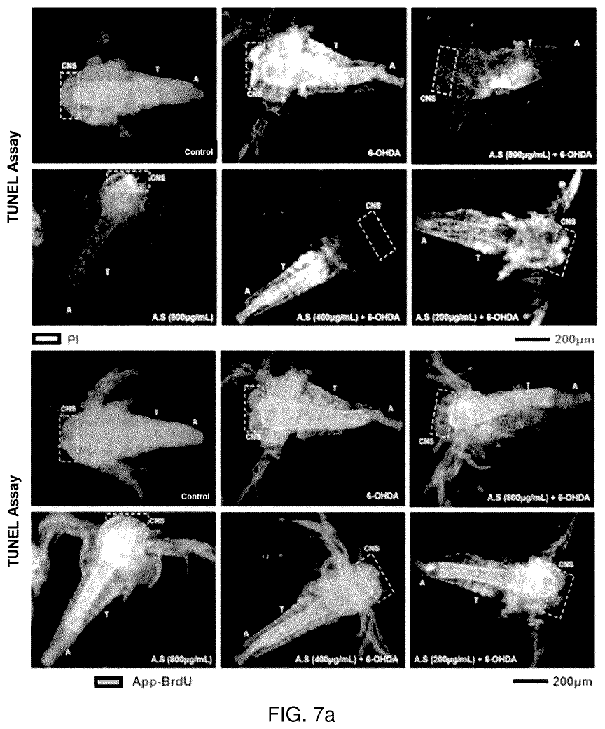

As shown in FIGS. 5a and 5b, 6-OHDA treatment increased ROS levels, leading to upregulation of MAOB and Cas3 expression in neuroblastoma cells. However, when 6-OHDA and Allium senescens extract were co-treated, the upregulation of MAOB and Cas3 was suppressed. Notably, at 6-OHDA concentrations of 80 μM and 100 μM, co-treatment with Allium senescens extract resulted in: MAOB expression being reduced by more than threefold; and Cas3 expression being reduced by more than 1.8-fold.

These findings confirm that Allium senescens extract effectively inhibits the expression of apoptotic genes MAOB and Cas3.

### Example 6: Assay for Oxidative Apoptosis-Related Marker Expression Regulation by Allium senescens Extract

The inventors analyzed the effect of Allium senescens extract on the expression of apoptosis markers in neuroblastoma cells by measuring the mRNA levels of the genes listed in Table 1. Neuroblastoma cells cultured according to Example 1-1 were divided into the following groups: i) Control group (Con): No treatment, ii) Ex group: Treated with 500 μg/mL Allium senescens extract, iii) 6OHDA(40 μM) group: Treated with 40 μM 6-OHDA, iv) 6OHDA(40 μM)+EX group: Pre-treated with 500 μg/mL Allium senescens extract, followed by treatment with 40 μM 6-OHDA, v) 6OHDA(80 μM) group: Treated with 80 μM 6-OHDA, vi) 6OHDA(80 μM)+EX group: Pre-treated with 500 μg/mL Allium senescens extract, followed by treatment with 80 μM 6-OHDA, vii) 6OHDA(100 μM) group: Treated with 100 μM 6-OHDA, viii) 6OHDA(100 μM)+EX group: Pre-treated with 500 μg/mL Allium senescens extract, followed by treatment with 100 μM 6-OHDA. Total RNA was extracted from the cells using RiboEx reagent (GeneAll, Korea). When applying both Allium senescens extract and 6-OHDA thereto, the group was treated with Allium senescens extract for 1 hour before exposure to 6-OHDA for 1 hour.

The extracted RNA was reverse-transcribed into cDNA using Maxime RT PreMix (iNtRON, Korea), and quantitative PCR (qPCR) was performed using the primers listed in Table 1 with the following cycle parameters: 95 °C for 1 minute, 59 °C for 35 seconds (35 cycles), and 72 °C for 1 minute (35 cycles). The expression levels of target genes were normalized to GAPDH (glyceraldehyde 3-phosphate dehydrogenase) and expressed relative to the control group, as shown in FIG. 6.

TABLE 1

| | | Nucleotide Sequence | SEQ ID |
|---|---|---|---|
| Gene | Direction | (5′ to 3′) | NO. |
| AKT | Forward | GGCTGCCAAGTGTCAAATCC | 1 |
| | Reverse | AGTGCTCCCCCACTTACTTG | 2 |
| NF_{K} B-P50 | Forward | CGGAGCCCTCTTTCACAGTT | 3 |
| | Reverse | TTCAGCTTAGGAGCGAAGGC | 4 |
| NF_{K} B-P52 | Forward | AGGTGCTGTAGCGGGATTTC | 5 |
| | Reverse | AGAGGCACTGTATAGGGCAG | 6 |
| Bcl2 | Forward | CTGCTGACATGCTTGGAAAA | 7 |
| | Reverse | ATTGGGCTACCCCAGCAATG | 8 |
| BAX | Forward | AGCGCTCCCCCACTTACTTG | 9 |
| | Reverse | GACAGGGACATCAGTCGCTT | 10 |
| CytC | Forward | ATGAATGACCACTCTAGCCA | 11 |
| | Reverse | ATAGAAACAGCCAGGACCGC | 12 |
| GAPDH | Forward | GTGGTCTCCTCTGACTTCAACA | 13 |
| | Reverse | CTCTTCCTCTTGTGCTCTTGCT | 14 |

The results demonstrated that Allium senescens extract inhibited the expression of apoptosis markers in neuroblastoma cells. When cells were treated with 10 μM 6-OHDA, the upregulation of anti-apoptotic markers AKT and BCL-2 was suppressed, while apoptotic markers Bax and CytC (cytochrome C) were significantly increased. However, in cells treated with Allium senescens extract, the expression of anti-apoptotic markers AKT, P50 (nuclear factor kappa-light-chain-enhancer of activated B cells-P50, NFκB-P50), P52, and BCL-2 was upregulated, while the upregulation of apoptotic markers Bax and CytC was inhibited (FIG. 6).

These findings confirm that Allium senescens extract promotes the expression of anti-apoptotic genes while inhibiting the expression of apoptotic genes.

### Example 7: Protection Effect of Allium senescens Extract Against Oxidative Stress in Brine Shrimp

### Example 7-1: Brine Shrimp Culturing

To evaluate the in vivo neuroprotective effects of Allium senescens extract, brine shrimp (Artemia franciscana) were used. Brine shrimp are invertebrates widely used in toxicity analyses for chemicals, water pollution, and natural products. Brine shrimp nervous system development is activated in the early nauplius stage, and these neurons exhibit high sensitivity to toxic chemicals. Parkinson's disease results from the degeneration of dopaminergic neurons in the substantia nigra. The equivalent region in brine shrimp is the mushroom body (MB) of the protocerebrum.

Brine shrimp cysts (1 g) were cultured in 3 L of artificial seawater (pH 8.2, 28 °C) with aeration for 2 days. Using a submarine assay, initial nauplii were classified, with subsequent establishment of treatment doses of 6-OHDA and the extract. For whole-mount immunohistochemistry and terminal deoxynucleotidyl transferase dUTP nick end labeling assay (TUNEL assay), the nauplius larvae were divided into the following groups: i) Control group (Con): No treatment, ii) 6OHDA(100 μM) group: Treated with 100 μM 6-OHDA, iii) Ext(800 μg) group: Treated with 800 μg/mL Allium senescens extract, iv) Ext(800 μg)+6OHDA group: Pre-treated with 800 μg/mL Allium senescens extract, followed by treatment with 100 μM 6-OHDA, v) Ext(400 μg)+6OHDA group: Pre-treated with 400 μg/mL Allium senescens extract, followed by treatment with 100 μM 6-OHDA, and vi) Ext(200 μg)+6OHDA group: Pre-treated with 200 μg/mL Allium senescens extract, followed by treatment with 100 μM 6-OHDA. When applying both Allium senescens extract and 6-OHDA thereto, the group was treated with Allium senescens extract for 30 min before exposure to 6-OHDA for 30 min.

### Example 7-2: Inhibitory Effect on Oxidative Stress in Central and Ventral Nervous System of Brine Shrimp

For terminal deoxynucleotidyl transferase dUTP nick end labeling assay (TUNEL assay), all the nauplius samples were stained with a TUNEL Assay Kit (Thermo Fisher Scientific, Waltman, USA). Fluorescence intensity was measured using a fluorescence microscope (Eclipse Ts-2, Nikon, Japan) and NIS-elements V5.11 imaging software (Nikon, Japan).

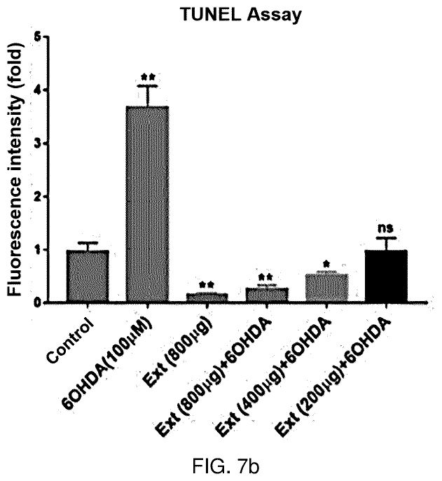

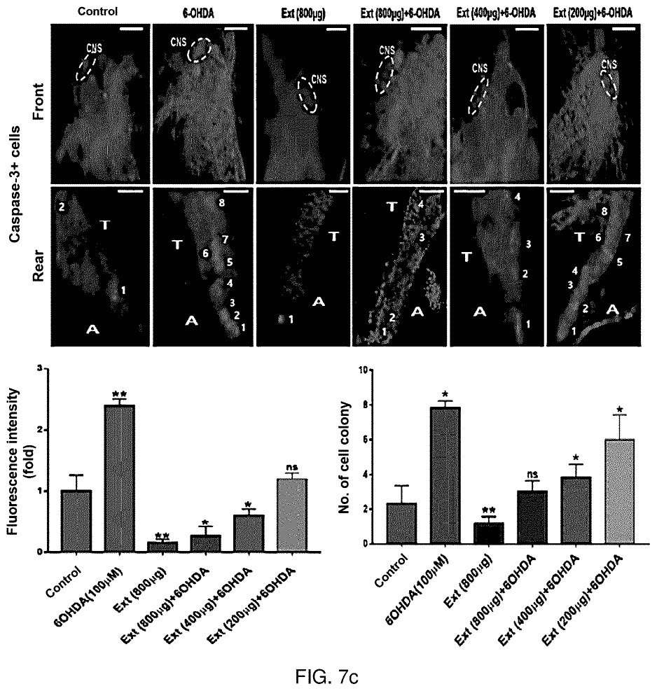

The results, as shown in FIG. 7a, demonstrated that 6-OHDA exposure induced neuronal apoptosis in brine shrimp during their developmental process. Following 6-OHDA exposure, the neurons of the central nervous system (CNS) and segmented neurons of the ventral nervous system (VNS) in nauplius larvae were strongly stained with PI (red) and apo-BruU (green), with fluorescence intensity approximately 3.7 times higher than that of the control group. In contrast, in the group treated with 800 μg/mL of Allium senescens extract, neuronal apoptosis was inhibited in both the CNS and VNS, with a more pronounced effect observed in the CNS of the nauplius larvae. Furthermore, in the thoracic region, apoptosis was inhibited in the segmented neurons of the group treated with 800 μg/mL of Allium senescens extract.

### Example 7-3: Effect of Extract on Mitochondrial Apoptosis in Central and Ventral Nervous System Cells of Brine Shrimp

To perform whole-mount immunohistochemistry, all nauplius samples were fixed in 4% paraformaldehyde for 4 hours and treated with 0.02% Tween 20 for 15 minutes. The treated samples were stained with Alexa Fluor 680-anti-Cas3 (Santa Cruz Biotechnology, USA) for 4 days. Fluorescence intensity was assessed using a fluorescence microscope (Eclipse Ts-2, Nikon, Japan) and analyzed with NIS-elements V5.11 imaging software (Nikon, Japan).

The results, as shown in FIG. 7c, revealed that treatment with 100 μM 6-OHDA induced mitochondrial apoptosis in cell colonies within the thoracic and abdominal regions, as well as in the CNS. The apoptosis rate in cells exposed to 6-OHDA was 2.3 times higher than that of the control group (Con). However, in cells treated with 800 μg/mL of Allium senescens extract, the apoptosis rate was 11.5 times lower than in the CNS exposed to 6-OHDA. Additionally, while the number of ganglia and fluorescence intensity increased upon 6-OHDA treatment, interestingly, in the group treated with Allium senescens extract, the number of stained cell colonies significantly decreased despite 6-OHDA exposure.

The results from Examples 7-2 and 7-3 confirmed that treatment with 6-OHDA strongly induced apoptosis in the protocerebrum, particularly in the mushroom body, of brine shrimp. In contrast, when co-treated with Allium senescens extract, neuronal apoptosis in brine shrimp was effectively inhibited, demonstrating the potential of Allium senescens extract as a therapeutic agent for neurodegenerative diseases, particularly Parkinson's disease. Notably, the anti-apoptotic effect of Allium senescens extract was more pronounced in the central nervous system.

Although the present disclosure has been described in detail with reference to the specific features, it will be apparent to those skilled in the art that this description is only of a preferred embodiment thereof, and does not limit the scope of the present disclosure.

1. A pharmaceutical composition for treatment of neurodegenerative disease, comprising Allium senescens extract as an active ingredient.

2. The pharmaceutical composition of claim 1, wherein the Allium senescens extract is obtained by extraction using a solvent selected from the group consisting of water, an organic solvent, and a mixture thereof.

3. The pharmaceutical composition of claim 2, wherein the organic solvent is at least one selected from the group consisting of lower alcohols of 1 to 4 carbon atoms, petroleum ether, hexane, benzene, chloroform, methylene chloride, ether, ethyl acetate, and acetone.

4. The pharmaceutical composition of claim 1, wherein the composition reduces reactive oxygen species (ROS) levels in neuronal cells.

5. The pharmaceutical composition of claim 1, wherein the composition protects mitochondrial activity in neuronal cells.

6. The pharmaceutical composition of claim 1, wherein the composition inhibits mitochondrial apoptosis in neuronal cells.

7. The pharmaceutical composition of claim 1, wherein the composition inhibits the expression of monoamine oxidase B (MAOB) or caspase-3 (Cas3) in neuronal cells.

8. The pharmaceutical composition of claim 1, wherein the composition inhibits the expression of apoptosis markers selected from the group consisting of monoamine oxidase B (MAOB), caspase-3 (Cas3), Bax, cytochrome C (CytC), and combinations thereof.

9. The pharmaceutical composition of claim 1, wherein the composition promotes the expression of anti-apoptotic markers selected from the group consisting of AKT (Protein kinase B), P50 (nuclear factor kappa-light-chain-enhancer of activated B cells-P50, NF-κB-P50), P52, BCL-2 (BCL2 apoptosis regulator), and combinations thereof.

10. A food composition for the alleviation of neurodegenerative disease, comprising Allium senescens extract as an active ingredient.

11. A method for treating or alleviating neurodegenerative disease, comprising:
administering an effective amount of a composition comprising Allium senescens extract as an active ingredient to a subject in need thereof.
